(19) United States
(12) Patent Application Publication
(10) Pub. No.: US 2026/0355523 A1
(43) Pub. Date: Nov. 12, 2026

(54) MULTIPLE ELECTRODE ARRANGEMENT FOR THERAPY

(71) Applicant: Medtronic, Inc., Minneapolis, MN (US)

(72) Inventors: Amy E. Thompson-Nauman, Ham Lake, MN (US); Linnea R. Lentz, Stacy, MN (US)

(21) Appl. No.: 19/125,040

(22) PCT Filed: Oct. 12, 2023
(86) PCT No.: PCT/IB2023/060311

§ 371 Date: Apr. 28, 2025

Related U.S. Application Data
(60) Provisional application No. 63/381,377, filed on Oct. 28, 2022.

(51) Int. Cl. A61N 1/362; A61N 1/05; A61N 1/39

(52) U.S. Cl. CPC A61N 1/3627; A61N 1/0587; A61N 1/39622

(57) ABSTRACT

In some examples, a system includes one or more leads comprising a plurality of electrodes, wherein the one or more leads are configured to position the plurality of electrodes in a plurality of extracardiac locations to deliver cardiac pacing to a right atrium (RA), a right ventricle (RV), a left atrium (LA), and/or a left ventricle (LV) of a heart of a patient. The system may further include an implantable medical device (IMD) coupled to the one or more leads. The IMD device may be configured to deliver the cardiac pacing via the plurality of electrodes.

This application claims the benefit of U.S. Provisional Patent Application Ser. No. 63/381,377, filed Oct. 28, 2022, the entire content of which is incorporated herein by reference.

### TECHNICAL FIELD

The present application relates to cardiac sensing and therapy and, more particularly, configurations of implantable systems for improved defibrillation, sensing, and/or pacing capabilities in extracardiac applications.

### BACKGROUND

Implantable systems, such as pacemakers with or without cardioversion or defibrillation capabilities, may treat cardiac dysfunction, such as bradycardia, tachyarrhythmia, and heart failure. Such implantable systems may include electrical devices configured to deliver therapy via electrodes, often carried by one or more implantable medical leads. Therapy for tachyarrhythmias may include shocks and/or anti-tachycardia pacing (ATP). The implantable systems may also be configured to deliver cardiac pacing to, for example, treat bradyarrhythmia or for cardiac resynchronization therapy (CRT).

CRT is delivered to improve cardiac electromechanical function. CRT may help enhance cardiac output by resynchronizing the electromechanical activity of the ventricles of the heart. CRT typically includes delivery of pacing via endocardial leads to one or both of the ventricles to synchronize their contracts. Ventricular desynchrony may occur in patients that suffer from congestive heart failure (CHF).

Owing to the inherent surgical risks in attaching and replacing implantable medical leads directly within or on the heart, subcutaneous implantable systems have been devised, in which the implantable system and leads are located subcutaneously outside of the thorax. It has also been proposed that the distal portion of a lead of an implantable system may be implanted within the thorax, e.g., substernally.

Implantable medical leads are also used to monitor and/or deliver therapies to tissues other than the heart. Implantable medical leads may be used to position one or more electrodes within or near target nerves, muscles, or organs to deliver electrical stimulation to such tissues. Implantable medical leads may be used to position one or more sensors within or near target tissue to monitor biological signals from such tissues. As examples, implantable medical leads may be positioned in the epidural space to deliver spinal cord stimulation, or proximate to other nerves, such as pelvic nerves or renal nerves, to deliver neurostimulation to the nerves.

### SUMMARY

Electrical stimulation therapy using extracardiac leads includes delivery of electrical pulses directed to a given chamber of the heart via electrodes on the leads. This disclosure describes implantable medical systems, which may include implantable extracardiac leads with electrodes configured to sense and pace multiple regions of the heart. Extracardiac locations for lead placement may include extravascular locations (i.e., outside of the vasculature and/or vasculature system) and/or locations in extracardiac vessels within the thorax (e.g., including but not limited to the internal thoracic vein (ITV), the intercostal veins, the superior epigastric vein, the azygos veins, the hemiazygos veins, and accessory hemiazygos veins). For example, this disclosure describes leads configured to deliver cardiac pacing to a right atrium (RA), a right ventricle (RV), a left atrium (LA) and/or a left ventricle (LV) of a heart of a patient by placement of the leads in respective extravascular regions or within extracardiac vessels, e.g., both anterior and posterior of the heart. In this manner, by capturing the RA, RV, and LV, cardiac resynchronization therapy (CRT) may be achieved. CRT may also be achieved by capturing the LA, LV, and RV.

Although described primarily in the context of implantable medical devices having one or more leads, the techniques of this disclosure may be applied using leadless pacing devices. Leadless pacing devices may also be placed in various extracardiac regions to facilitate capture and/or sensing of the RA, RV, and LV. Leadless pacing devices may be used alone, or in combination with devices having one or more leads. The present disclosure discusses the context of ICD systems, as well as systems other than ICD systems, including, but not limited to, bradycardia or cardiac resynchronization therapy (CRT) pacemaker systems, which may not be configured to deliver antitachyarrhythmia shocks.

In one example, a system includes one or more leads and an implantable medical device (IMD) coupled to the one or more leads. The one or more leads include a plurality of electrodes. The one or more leads are configured to position the plurality of electrodes in a plurality of extracardiac locations to deliver cardiac pacing to a right atrium (RA), a right ventricle (RV), and a left ventricle (LV) of a heart of a patient. The IMD device is configured to deliver the cardiac pacing via the plurality of electrodes.

In another example, a system includes one or more leads, an implantable medical device (IMD) coupled to the one or more leads, and one or more leadless pacing devices. The one or more leads include a plurality of electrodes. The one or more leads are configured to position the plurality of electrodes at a plurality of extracardiac locations to deliver cardiac pacing to a first subset of a right atrium (RA), a right ventricle (RV), and a left ventricle (LV) of a heart of a patient. The IMD device is configured to deliver the cardiac pacing via the plurality of electrodes. The one or more leadless pacing devices may be configured to deliver cardiac pacing to a second subset of the RA, the RV, the LV.

In another example, a method includes positioning, by one or more leads comprising a plurality of electrodes, the plurality of electrodes to deliver cardiac pacing to a right atrium (RA), a right ventricle (RV), and a left ventricle (LV) of a heart of a patient. The method further includes delivering, by an implantable medical device (IMD) coupled to the one or more leads, cardiac pacing to the RA, the RV, and the LV of the heart of the patient via the plurality of electrodes positioned in a plurality of extracardiac locations by the one or more leads.

In another example, a method includes positioning, by one or more leads comprising a plurality of electrodes, the plurality of electrodes to deliver cardiac pacing to a first subset of a right atrium (RA), a right ventricle (RV), and a left ventricle (LV) of a heart of a patient. The method further includes delivering, by an implantable medical device (IMD) coupled to the one or more leads, cardiac pacing to the first subset of the RA, the RV, and the LV of the heart of the patient via the plurality of electrodes positioned at a plurality of extracardiac locations by the one or more leads. The method further includes delivering, by one or more leadless pacing devices, cardiac pacing to a second subset of the RA, the RV, and the LV of a heart of a patient.

In another example, a system includes a plurality of pacing devices configured to be positioned in a plurality of extracardiac locations. The plurality of leadless pacing devices are configured to pace a right atrium (RA), a right ventricle (RV), or a left ventricle (LV), or any combination thereof of a heart of a patient.

In another example, a system includes one or more extracardiac leads including a plurality of electrodes. The one or more leads includes a first extracardiac lead configured to position a first subset of the plurality of electrodes to deliver cardiac pacing to a right atrium (RA) and a right ventricle (RV) of a heart of a patient. The one or more leads includes a second extracardiac lead configured to position a second subset of the plurality of electrodes to deliver cardiac pacing to a left ventricle (LV). The plurality of electrodes includes a plurality of electrodes configured to deliver cardiac pacing. The one or more leads comprise one or more electrodes configured to deliver antitachyarrhythmia shocks. The system includes an implantable medical device (IMD) coupled to the one or more leads. The IMD device is configured to deliver the cardiac pacing via the plurality of electrodes configured to deliver cardiac pacing and deliver antitachyarrhythmia shocks via the one or more electrodes configured to deliver the antitachyarrhythmia shocks. The first lead is configured to be positioned anterior relative to the heart of the patient, and the second lead is configured to be positioned posterior relative to the heart of the patient.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

FIG. 1 is a conceptual drawing illustrating a front view of a patient implanted with an example extracardiac implantable medical device system.

FIG. 2 is a conceptual drawing illustrating a side view of a patient implanted with an example extracardiac implantable medical device system.

FIG. 3 is a conceptual drawing illustrating a transverse view of a patient implanted with an example extracardiac implantable medical device system.

FIG. 4 is a functional block diagram of an example configuration of electronic components of an example IMD.

FIG. 5 is a conceptual drawing illustrating a front view of a patient implanted with an example extracardiac implantable medical device system.

FIG. 6 is a conceptual drawing illustrating a front view of a patient implanted with an example extracardiac implantable medical device system showing vertical leads.

FIG. 7 is a conceptual drawing illustrating a front view of a patient implanted with an example extracardiac implantable medical device system showing vertical and/or horizontal leads.

FIG. 8 is a conceptual drawing illustrating a front view of a patient implanted with an example extracardiac implantable medical device system including multiple leadless pacing devices.

FIG. 9 is a conceptual drawing illustrating a front view of a patient implanted with an example extracardiac implantable medical device system along with one or more leadless pacing devices.

FIG. 10 is a flow chart of an example technique for delivering therapy via an example extracardiac implantable medical device system.

FIG. 11 is a flow chart of an example technique for implanting one or more leads within a patient.

Like reference characters denote like elements throughout the description and figures.

### DETAILED DESCRIPTION

In accordance with the techniques of the disclosure, a system is configured to provide cardiac resynchronization therapy (CRT) by providing pacing to multiple regions of the heart from extracardiac electrodes. For example, the system may include extracardiac leads configured to deliver cardiac pacing to a right atrium (RA), a right ventricle (RV), and a left ventricle (LV).

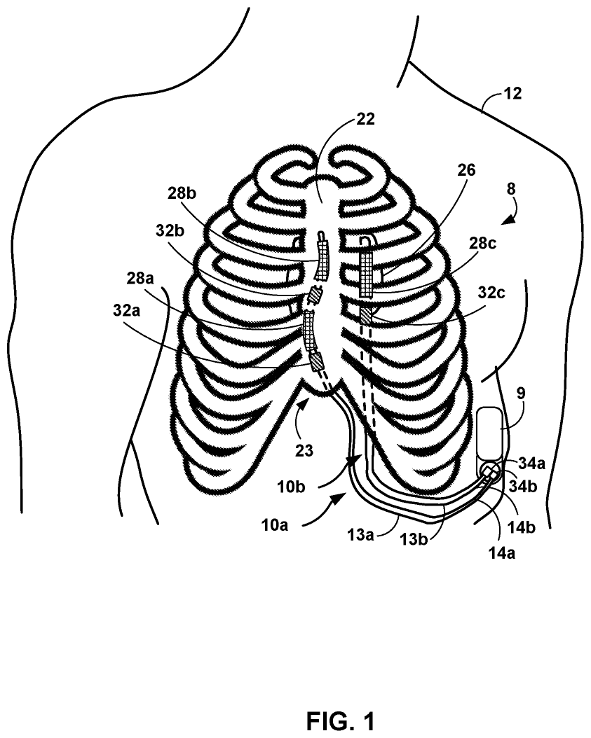

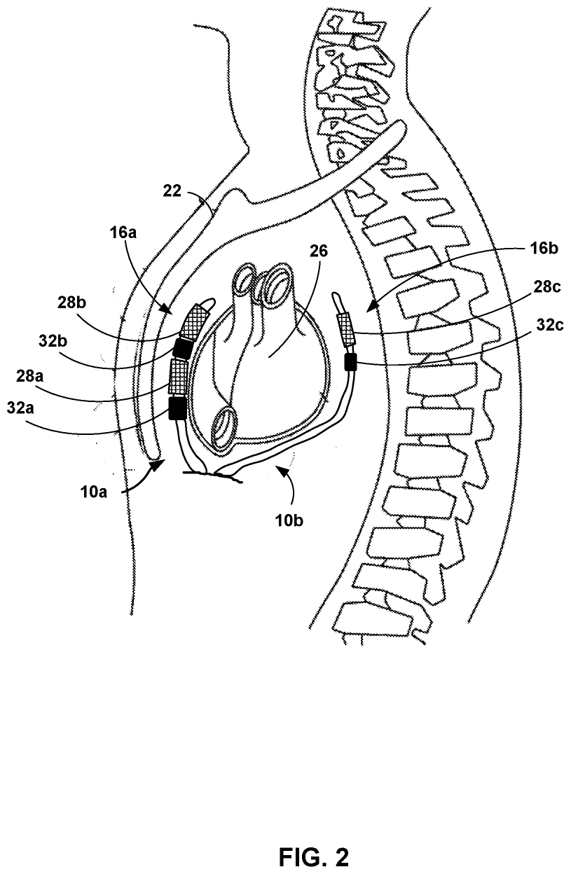

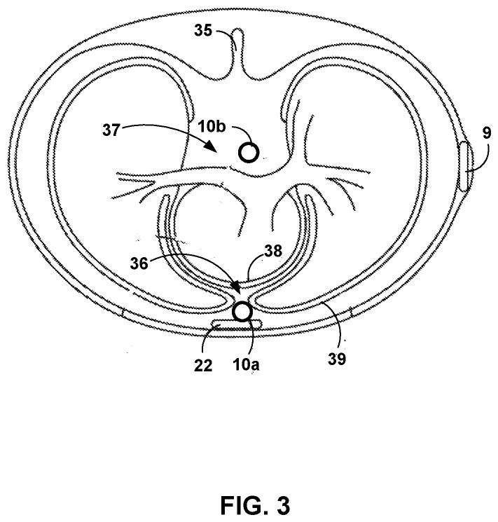

FIG. 1 is a front view of a patient 12 with an implantable medical device (IMD) system 8 implanted intrathoracically. Referring to the drawings in which like reference designators refer to like elements, FIGS. 1-3 shows conceptual diagrams illustrating various views of an example extracardiac IMD system 8. IMD system 8 includes an IMD 9 connected to a first lead 10a and a second lead 10b (collectively “implantable leads 10” or “one or more leads 10”). The one or more leads 10 may be implantable medical leads. FIG. 1 is a front view of a patient implanted with extracardiac IMD system 8. FIG. 2 is a side view of the patient implanted with extracardiac IMD system 8. FIG. 3 is a transverse view of the patient implanted with extracardiac IMD system 8.

IMD 9 may include a housing that forms a hermetic seal that protects components of the IMD 9. The housing of IMD 9 may be formed of a conductive material, such as titanium or titanium alloy, which may function as a housing electrode (sometimes referred to as a can electrode). In some embodiments, IMD 9 may be formed to have or may include a plurality of electrodes on the housing. IMD 9 also includes a connector assembly (also referred to as a connector block or header) that includes electrical feedthroughs through which electrical connections are made between conductors of one or more leads 10 and electronic components included within the housing of IMD 9. As will be described in further detail herein, the housing may house one or more processors, memories, transmitters, receivers, sensors, sensing circuitry, therapy circuitry, power sources and other appropriate components. The housing may be configured to be implanted in a patient, such patient 12. Alternatively, the housing may be external to the patient and connect to a proximal end of the lead that extends out of the body of the patient, e.g., through an incision.

IMD 9 is implanted extrathoracically on the left side of the patient, e.g., under the skin and outside the ribcage (subcutaneously or submuscularly). IMD 9 may, in some instances, be implanted between the left posterior axillary line and the left anterior axillary line of the patient. IMD 9 may, however, be implanted at other extrathoracic locations on the patient, implanted in an intrathoracic location, or not implanted at all in the case of an external pacemaker.

An IMD, such as IMD 9, is coupled to one or more leads 10. Lead 10a and lead 10b may be configured to be placed at a plurality of extracardiac locations, including extravascular locations (i.e., outside of the vasculature and/or vasculature system) and/or locations in extracardiac vessels within the thorax. Lead 10a may include elongated lead body 13a having a distal portion 16a and lead 10b may include elongated lead body 13b having a distal portion 16b. The one or more leads 10 may be sized to be implanted in an extravascular location proximate the heart, e.g., intrathoracically, as illustrated in FIGS. 1-3, or extrathoracically. For example, the one or more leads 10 may extend extrathoracically under the skin and outside the ribcage (e.g., subcutaneously, submuscularly, and/or supradiaphragmatically) from IMD 9 toward the center of the torso of the patient, for example, toward the xiphoid process 23 of the patient. At a position proximate xiphoid process 23, the lead body 13a and/or lead body 13b may bend or otherwise turn and extend superiorly. The bend may be pre-formed and/or lead body 13a or lead body 13b may be flexible to facilitate bending.

In the example illustrated in FIGS. 1-3, lead body 13a extends superiorly intrathoracically anterior to heart 26 and underneath sternum 22, in a direction substantially parallel to sternum 22. In this manner, distal portion 16a of lead 10a may reside in an extravascular intrathoracic substernal location such that distal portion 16a of lead 10a extends superior along the posterior side of the sternum 22 substantially within the anterior portion of mediastinum 36. Lead body 13b extends superiorly intrathoracically posterior to heart 26 and anterior to spinal column 35. In this manner, distal portion 16b of lead 10b may reside in an extravascular intrathoracic posterior portion of mediastinum 37. Lead 10a and lead 10b may share a common point of entry into the mediastinum. Alternatively, lead 10a and 10b may have separate points of entry in the respective portions of the mediastinum. In some examples, lead 10b may be the only lead implanted in a patient, and lead 10b may be positioned in a posterior mediastinum 37 (such as to achieve posterior mediastinal pacing, sensing or defibrillation when the anterior mediastinum is avoided and/or inaccessible for electrode implantation).

Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic artery, and the internal thoracic vein (ITV). Posterior mediastinum 37 may be viewed as being bounded laterally by pleurae 39, posteriorly by vertebral column 35, and anteriorly by pericardium 38. Posterior mediastinum 37 includes part of the descending aorta, the azygos and the two hemiazygos veins, the vagus and splanchnic nerves, the esophagus, the thoracic duct, and some lymph glands. The mediastinum may be bounded inferiorly by the diaphragm.

Lead body 13a may extend superiorly extrathoracically (instead of intrathoracically), e.g., either subcutaneously or submuscularly above the ribcage/sternum. In either the extrathoracic or intrathoracic examples, lead 10a may be implanted at other locations, such as over or under the sternum, offset to the right or left of the sternum, angled lateral from the proximal or distal end of the sternum, or the like. In some examples, lead 10a may be implanted in an extravascular intercostal location (i.e., between the ribs). In some examples, lead 10a may be implanted within an extracardiac vessel within the thorax, such as the ITV, the intercostal veins, or the superior epigastric vein. In some examples, distal portion 16a of lead 10a may be oriented differently than is illustrated in FIGS. 1-3, such as orthogonal or otherwise transverse to sternum 22 and/or inferior to heart 26. In such examples, distal portion 16a of lead 10a may be at least partially within anterior mediastinum 36. Lead 10b may be implanted at other locations. In some examples, one or more leads 10 may be implanted within an extracardiac vessel within the thorax, such as the azygos, hemiazygos, and accessory hemiazygos veins. In some examples, lead 10b may be implanted in an extravascular intercostal location (i.e., between the ribs).

Lead body 13a and lead body 13b may have a generally tubular or cylindrical shape and may define a diameter of approximately 3-9 French (Fr). However, lead bodies of less than 3 Fr and more than 9 Fr may also be utilized. In another configuration, lead body 13a and lead body 13b may have a flat, ribbon, or paddle shape with solid, woven filament, or metal mesh structure, along at least a portion of the length of the lead body 13a and lead body 13b. In such an example, the width across lead body 13a and lead body 13b may be between 1-3.5 mm. Other lead body designs may be used without departing from the scope of this application.

Lead body 13a and lead body 13b may be formed from a non-conductive material, including silicone, polyurethane, fluoropolymers, mixtures thereof, and other appropriate materials, and shaped to form one or more lumens (not shown), however, the techniques are not limited to such constructions. Distal portion 16a and distal portion 16b may be fabricated to be biased in a desired configuration, or alternatively, may be manipulated by the user into the desired configuration. For example, the distal portion 16a and distal portion 16b may be composed of a malleable material such that the user can manipulate the distal portion into a desired configuration where it remains until manipulated to a different configuration.

Lead body 13a may include a proximal end 14a having a connector 34a configured to couple to IMD 9 and a distal portion 16a which include electrodes configured to deliver electrical energy to the heart or sense electrical signals of the heart. Similarly, lead body 13b may include a proximal end 14b having a connector 34b configured to couple to IMD 9 and a distal portion 16b which include electrodes configured to deliver electrical energy to the heart or sense electrical signals of the heart. The connectors of lead bodies 13a and 13b may be industry standard connectors (e.g., IS1, DF1, IS4, DF4, or the like) or propriety connectors. In some instances, distal portion 16a and distal portion 16b may be anchored to a desired position within the patient, for example, substernally or subcutaneously by, for example, suturing distal portion 16a and distal portion 16b to the patient's musculature, tissue, or bone at the xiphoid process entry site. In some examples, distal portion 16a and distal portion 16b may be anchored to the patient or through the use of rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements.

Distal portion 16a and distal portion 16b may include one or more defibrillation electrodes configured to deliver an anti-tachyarrhythmia, e.g., cardioversion/defibrillation, shock to heart 26 of patient 12. In some examples, one or both of distal portion 16a and distal portion 16b include a plurality of defibrillation electrodes spaced a distance apart from each other along the length of distal portion 16. In the example illustrated by FIGS. 1-3, distal portion 16a of lead 10a includes two defibrillation electrodes 28a and 28b, and distal portion 16b of lead 10b includes one defibrillation electrodes 28c (collectively, “defibrillation electrodes 28”). In other examples, each of distal portions 16 may include one defibrillation electrode 28, each of distal portions 16 may include more than one defibrillation electrode 28, or one of distal portions 16, such as distal portion 16b, may not include a defibrillation electrode 28. In some examples, defibrillation electrodes 28 may be electrically separate electrodes, or electrically common segments of a single defibrillation electrode.

Defibrillation electrodes 28 may be disposed around or within the lead body 13a and lead body 13b of the distal portions 16a and 16b, or alternatively, may be embedded within the wall of the lead body 13a and lead body 13b. In one configuration, defibrillation electrodes 28 may be coil electrodes formed by a conductor. The conductor may be formed of one or more conductive polymers, ceramics, metal-polymer composites, semiconductors, metals or metal alloys, including but not limited to, one of a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole, and other polymers. In another configuration, each of defibrillation electrodes 28 may be a flat ribbon electrode, a paddle electrode, a braided or woven electrode, a mesh electrode, a directional electrode, a patch electrode or another type of electrode configured to deliver a cardioversion/defibrillation shock to heart 26 of patient 12.

Defibrillation electrodes 28 may be electrically connected to one or more conductors, which may be disposed in a lumen defined by a body wall of lead body 13a and lead body 13b or in one or more insulated lumens (not shown) defined by lead body 13a and lead body 13b. In an example configuration, each of defibrillation electrodes 28 is connected to a common conductor such that a voltage may be applied simultaneously to all defibrillation electrodes 28 to deliver an anti-tachyarrhythmia shock to heart 26. In other configurations, defibrillation electrodes 28 may be attached to separate conductors such that each defibrillation electrode 28 may apply a voltage independent of the other defibrillation electrodes 28. In this case, IMD 9 or one or more leads 10 may include one or more switches or other mechanisms to electrically connect the defibrillation electrodes together to function as a common polarity electrode such that a voltage may be applied simultaneously to all defibrillation electrodes 28 in addition to being able to independently apply a voltage.

The one or more leads 10 may include a plurality of electrodes, wherein the one or more leads 10 are configured to position the plurality of electrodes in a plurality of extracardiac locations to deliver cardiac pacing to a right atrium (RA), a right ventricle (RV), and a left ventricle (LV) of heart 26 of patient 12. In some examples, the cardiac pacing may include cardiac resynchronization therapy (CRT). Distal portions 16a and 16b may include one or more pacing and/or sensing electrodes configured to deliver pacing pulses to heart 26 and/or sense electrical activity of heart 26. Such electrodes may be referred to as pacing electrodes, sensing electrodes, or pace/sense electrodes. In the example illustrated by FIGS. 1-3, distal portion 16a includes two pace/sense electrodes 32a and 32b, and distal portion 16b includes one pace/sense electrode 32c (collectively, “pace/sense electrodes 32” or “plurality of electrodes 32”). However, in some examples, the one or more leads 10 may be configured to position pace/sense electrodes 32 to deliver cardiac pacing and/or monitor to the LA, the LV, and the RV to achieve CRT.

In the illustrated example of FIGS. 1-3, subsets of the plurality of electrodes 32, in which the subsets may include one or more electrodes, may be configured to pace certain regions of heart 26. For example, a first subset of electrodes, which may include one or more electrodes, may be configured to pace the RA and RV, and a second subset of electrodes, which may include one or more electrodes, may be configured to pace the LV. The one or more leads 10 may be configured to position the subsets of plurality of electrodes 32 to pace certain regions of heart 26. For example, a first lead (such as lead 10a) may be configured to position a first subset (such as electrodes 32a and 32b) of the plurality of electrodes 32 to deliver pacing to the RA and the RV. In some examples, a second lead (such as lead 10b) may be configured to position a second subset (such as electrode 32c) of the plurality of electrodes 32 to deliver pacing to the LV. In other examples, a first lead (such as lead 10a) may be configured to position a first subset of electrodes of the plurality of electrodes to deliver pacing to the RA, and a second lead (such as lead 10b) may be configured to position a second subset of the plurality of electrodes to deliver pacing to the LA and LV. In other examples, a first lead (such as lead 10a) may be configured to position a first subset of electrodes of the plurality of electrodes to deliver pacing to the RA and RV and a second lead (such as lead 10b) may be configured to position a second subset of the plurality of electrodes to deliver pacing to the LA and LV. Where a unique electrode separately senses the RA and LA, independent timing sequences may be coordinated for the RV and LV.

In the illustrated example of FIGS. 1-3, pace/sense electrode 32b is positioned between defibrillation electrodes 28a and 28b on lead 10a, e.g., within a gap between the defibrillation electrodes, and pace/sense electrode 32a is positioned more proximal along distal portion 16a than proximal defibrillation electrode 28a. In some examples, more than one electrode of the plurality of electrodes 32 may exist within the gap between defibrillation electrodes 28. In some examples, an electrode is additionally or alternatively located distal of the distalmost defibrillation electrode 28b. Additionally, pace/sense electrode 32c may be positioned distally or proximally compared to defibrillation electrodes 28c on lead 10b (although pace/sense electrode 32c is positioned proximally compared to defibrillation electrodes 28c on lead 10 in the example of FIG. 1). Additionally, lead 10b may include multiple pace/sense electrodes 28c and one or more defibrillation electrodes 28b.

Within the IMD system 8, IMD 9 may be configured to deliver cardiac pacing via plurality of electrodes 32. Plurality of electrodes 32 may be configured to deliver cardiac pacing. IMD 9 may be configured to deliver the antitachyarrhythmia shocks via the one or more electrodes configured to deliver the antitachyarrhythmia shocks. One or more leads 10 comprise one or more electrodes configured to deliver antitachyarrhythmia shocks. Plurality of electrodes 32 may be configured to deliver low-voltage electrical pulses to the heart or may sense a cardiac electrical activity, e.g., depolarization and repolarization of the heart. As such, plurality of electrodes 32 may be referred to as pace/sense electrodes 32. In one configuration, the plurality of electrodes 32 are ring electrodes. However, in other configurations, plurality of electrodes 32 may be any of a number of different types of electrodes, including ring electrodes, short coil electrodes, paddle electrodes, hemispherical electrodes, or directional electrodes. Each of the plurality of electrodes 32 may be the same or different types of electrodes as others of the plurality of electrodes 32. The plurality of electrodes 32 may be electrically isolated from an adjacent defibrillation electrode 28 by including an electrically insulating layer of material between the plurality of electrodes 32 and adjacent defibrillation electrodes 28. Each electrode of the plurality of electrodes 32 may have its own separate conductor such that a voltage may be applied to or sensed via each electrode independently from another electrode of the plurality of electrodes 32.

Electrodes 28 are referred to as defibrillation electrodes, and the plurality of electrodes 32 are referred to as pace/sense electrodes, because they may have different physical structures enabling different functionality. Defibrillation electrodes 28 may be larger, e.g., have greater surface area, than pace/sense electrodes 32 and, consequently, may be configured to deliver anti-tachyarrhythmia shocks that have relatively higher voltages than pacing pulses. The relatively smaller size of pace/sense electrodes 32 may provide advantages over defibrillation electrodes for delivering pacing pulses and sensing intrinsic cardiac activity, e.g., lower pacing capture thresholds and/or better sensed signal quality.

Nevertheless, a defibrillation electrode 28 may be used to deliver pacing pulses and/or sense electrical activity of the heart, such as in combination with a pace/sense electrode 32. In some examples, the one or more leads 10 may only include pace/sense electrodes 32 and not defibrillation electrodes 28, to achieve CRT, otherwise referred to as “CRT-P.”

Proximal end 14a of lead body 13a may include connector 34a to electrically couple lead 10a to IMD 9. Proximal end 14b of lead body 13b may include connector 34b to electrically couple lead 10b to IMD 9. IMD 9 may also include a connector assembly that includes electrical feedthroughs through which electrical connections are made between the one or more electrical contacts on connectors 34 of the one or more leads 10 and the electronic components included within the housing. The housing of IMD 9 may house one or more processors, memories, transmitters, receivers, sensors, sensing circuitry, therapy circuitry, power sources (e.g., capacitors and batteries), and/or other components. The components of IMD 9 may generate and deliver electrical therapy such as anti-tachycardia pacing, cardioversion or defibrillation shocks, post-shock pacing, bradycardia pacing, and/or CRT.

In some examples, an undulating configuration of distal portion 16a of lead 10a and the inclusion of electrode 32a and electrode 32b between defibrillation electrode 28a and defibrillation electrode 28b may provide a number of therapy vectors for the delivery of electrical therapy to the heart. For example, at least a portion of defibrillation electrodes 28a and 28b and one of pace/sense electrodes 32a and 32b may be disposed over the right ventricle, the right atrium, or any chamber of the heart, such that pacing pulses and anti-tachyarrhythmia shocks may be delivered to the heart. The housing of IMD 9 may be charged with or function as a polarity different than the polarity of the one or more defibrillation electrodes 28a and 28b and/or electrodes 32a and 32b such that electrical energy may be delivered between the housing and the defibrillation electrodes 28a and 28b and/or electrodes 32a and 32b to the heart. In some examples, defibrillation electrode 28c and a pace/sense electrode 32c may be disposed over the left ventricle such that pacing pulses and/or anti-tachyarrhythmia shocks may be delivered to the heart. In some examples, a defibrillation vector may be formed across the heart between defibrillation electrode 28c and at least one of defibrillation electrode 28a or 28b. In some examples, lead 10a including electrode 32a, electrode 32b, defibrillation electrode 28a, and defibrillation electrode 28b and lead 10b including defibrillation electrode 28c and pace/sense electrode 32c may provide a number of therapy vectors, including a transcardiac impedance vector. In some examples, lead 10a may only include pace/sense electrodes 32a and/or 32b. In some examples, lead 10a may only include defibrillation electrode 28a and/or 28b. In some examples lead 10b may only include pace/sense electrode 32c. In some examples, lead 10b may only include defibrillation electrode 28c.

Each defibrillation electrode of defibrillation electrodes 28 may have the same polarity as every other defibrillation electrode when a voltage is applied to it such that a shock may be delivered from all defibrillation electrodes together. In a first example, defibrillation electrodes 28a and 28b are electrically connected to a common conductor within lead body 13a and may have the same polarity. However, in other examples, defibrillation electrodes 28a and 28b may be coupled to separate conductors within lead body 13a and may therefore each have different polarities such that electrical energy may flow between defibrillation electrodes 28a and 28b, or between one of defibrillation electrodes 28a and 28b and one of pace/sense electrodes 32a or 32b or the housing electrode, to provide anti-tachyarrhythmia shock, pacing therapy, and/or to sense cardiac depolarizations. In this case, defibrillation electrodes 28a and 28b may still be electrically coupled together, e.g., via one or more switches within IMD 9, to have the same polarity.

In some examples, distal portion 16a of lead 10a and/or distal portion 16b of lead 10b may include one or more shields. The shield or shields may be configured to impede an electric field from delivery of an electrical therapy via an electrode, e.g., from a pacing pulse, in a direction from the electrode away from the heart, e.g., in an anterior or posterior direction. In this manner, the shield may reduce the likelihood that the electrical field will stimulate extracardiac tissue, such as sensory or motor nerves. Furthermore, the shield may direct the electrical field toward the heart, allowing lower energy level pacing pulses to capture the heart than may be required without the shield. Lower energy pacing pulses may also reduce the likelihood that pacing pulses delivered via the pacing electrode stimulate extracardiac tissue, and may result in less consumption of the power source of IMD 9 and, consequently, longer service life for IMD 9. The techniques of this disclosure may be applied to implantable systems other than IMD 9, including, but not limited to, bradycardia pacemaker systems. For example, a lead that does not include defibrillation electrodes may include one or more shields and may be used with a pacemaker system without defibrillation capabilities.

In accordance with the techniques of the disclosure, the pacing electrode of pace/sense electrodes 32 may be configured to decrease the pacing voltage threshold. For example, a conductive surface may be disposed on a shield and electrically coupled to the pacing electrode, which may reduce a resistance of the pacing electrode and/or expand an electric field generated by the pacing electrode. Reducing the resistance of the pacing electrode and/or expanding the electric field generated by the pacing electrode may reduce an amount of current used to generate a pacing pulse, which may decrease an amount of power used by IMD 9.

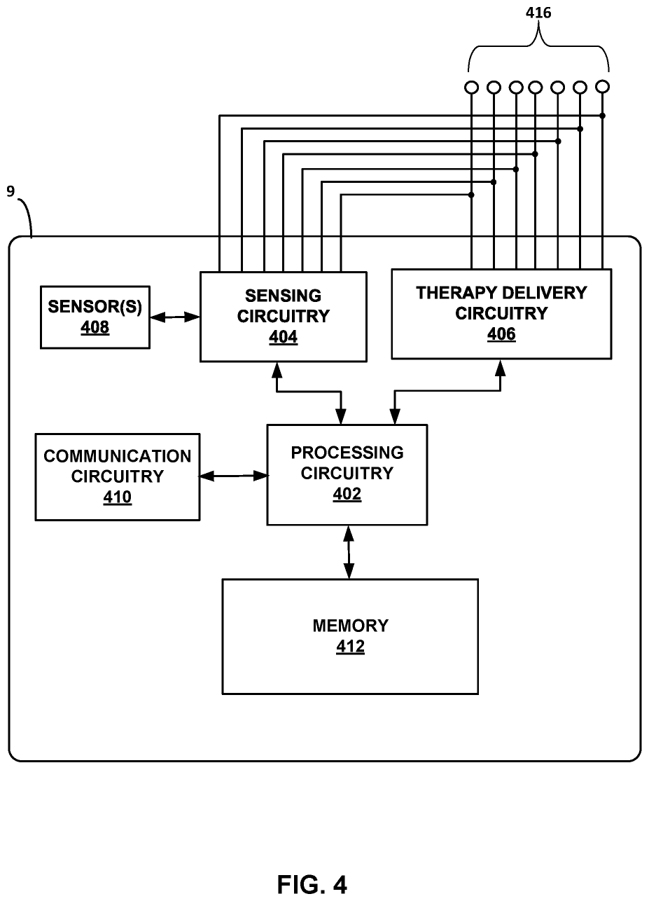

FIG. 4 is a functional block diagram of an example configuration of electronic components and other components of IMD 9. IMD 9 includes a processing circuitry 402, sensing circuitry 404, therapy delivery circuitry 406, sensors 408, communication circuitry 410, and memory 412. In some examples, IMD 9 may include more or fewer components.

The described circuitry and other components may be implemented together on a common hardware component or separately as discrete but interoperable hardware or software components. Depiction of different features is intended to highlight different functional aspects and does not necessarily imply that such circuitry and other components must be realized by separate hardware or software components. Rather, functionality associated with one or more circuitries and components may be performed by separate hardware or software components, or integrated within common or separate hardware or software components.

Sensing circuitry 404 may be electrically coupled to some or all of electrodes 416, which may correspond to any of the defibrillation, pace/sense, and housing electrodes described herein. Sensing circuitry 404 is configured to obtain signals sensed via one or more combinations of electrodes 416 and process the obtained signals.

The components of sensing circuitry 404 may be analog components, digital components or a combination thereof. Sensing circuitry 404 may, for example, include one or more sense amplifiers, filters, rectifiers, threshold detectors, analog-to-digital converters (ADCs) or the like. Sensing circuitry 404 may convert the sensed signals to digital form and provide the digital signals to processing circuitry 402 for processing or analysis. For example, sensing circuitry 404 may amplify signals from the sensing electrodes and convert the amplified signals to multi-bit digital signals by an ADC. Sensing circuitry 404 may also compare processed signals to a threshold to detect the existence of atrial or ventricular depolarizations (e.g., P-or R-waves) and indicate the existence of the atrial depolarization (e.g., P-waves) or ventricular depolarizations (e.g., R-waves) to processing circuitry 402. As shown in FIG. 4, IMD 9 may additionally include one or more sensors 408, such as one or more accelerometers, which may be configured to provide signals indicative of other parameters of a patient, such as activity or posture, to processing circuitry 402.

Processing circuitry 402 may process the signals from sensing circuitry 404 to monitor electrical activity of heart 26 of patient 12. Processing circuitry 402 may store signals obtained by sensing circuitry 404 as well as any generated EGM waveforms, marker channel data or other data derived based on the sensed signals in memory 412. Processing circuitry 402 may analyze the EGM waveforms and/or marker channel data to detect arrhythmias (e.g., bradycardia or tachycardia). In response to detecting the cardiac event, processing circuitry 402 may control therapy delivery circuitry 406 to deliver the desired therapy to treat the cardiac event, e.g., defibrillation shock, cardioversion shock, ATP, post shock pacing, bradycardia pacing, or CRT.

Therapy delivery circuitry 406 is configured to generate and deliver electrical therapy to heart 26. Therapy delivery circuitry 406 may include one or more pulse generators, capacitors, and/or other components capable of generating and/or storing energy to deliver as pacing therapy, defibrillation therapy, cardioversion therapy, CRT, other therapy or a combination of therapies. Therapy delivery circuitry 406 may be configured to generate and deliver pacing pulses with magnitudes and timings specified by processing circuitry 402. For delivery of CRT, for example, processing circuitry 402 may control therapy delivery circuitry to deliver pacing pulses according to one or more atrioventricular (AV) intervals from an intrinsic or paced atrial event, and/or one or more interventricular (VV) intervals from an intrinsic or paced ventricular event. The values of such intervals may be programmable by a user and/or variable based on physiological parameters sensed, e.g., via sensor(s) 408.

In some instances, therapy delivery circuitry 406 may include a first set of components configured to provide pacing therapy and a second set of components configured to provide defibrillation therapy. In some instances, therapy delivery circuitry 406 may utilize the same set of components to provide both pacing and defibrillation therapy. In still other instances, therapy delivery circuitry 406 may share some of the defibrillation and pacing therapy components while using other components solely for defibrillation or pacing. Processing circuitry 402 may control therapy delivery circuitry 406 to deliver the generated therapy to heart 26 via one or more combinations of electrodes 416. Although not shown in FIG. 4, IMD 9 may include switching circuitry configurable by processing circuitry 402 to control which of electrodes 416 is connected to therapy delivery circuitry 406 and sensing circuitry 404.

Communication circuitry 410 may include any suitable hardware, firmware, software or any combination thereof for communicating with another device, such as a clinician programmer, a patient monitoring device, or the like. For example, communication circuitry 410 may include appropriate modulation, demodulation, frequency conversion, filtering, and amplifier components for transmission and reception of data with the aid of an antenna.

The various components of IMD 9 may include any one or more processors, controllers, digital signal processors (DSPs), application specific integrated circuits (ASICs), field-programmable gate arrays (FPGAs), or equivalent discrete or integrated circuitry, including analog circuitry, digital circuitry, or logic circuitry. Processing circuitry 402 may include fixed function circuitry and/or programmable processing circuitry. The functions attributed to processing circuitry 402 herein may be embodied as software, firmware, hardware or any combination thereof.

Memory 412 may include computer-readable instructions that, when executed by processing circuitry 402 or other components of IMD 9, cause one or more components of IMD 9 to perform various functions attributed to those components in this disclosure.

Memory 412 may include any volatile, non-volatile, magnetic, optical, or electrical media, such as a random-access memory (RAM), read-only memory (ROM), non-volatile RAM (NVRAM), static non-volatile RAM (SRAM), electrically-erasable programmable ROM (EEPROM), flash memory, or any other non-transitory computer-readable storage media.

The leads and systems described herein may be used at least partially within the substernal space, e.g., within anterior or posterior mediastinum of patient, to provide an extracardiac IMD system. An implanter (e.g., a physician) may implant the distal portion of the lead intrathoracically using any of a number of implant tools, e.g., tunneling rod, sheath, or other tool that can traverse the diaphragmatic attachments and form a tunnel in the substernal location. For example, the implanter may create an incision near the center of the torso of the patient, e.g., and introduce the implant tool into the substernal location via the incision. The implant tool is advanced from the incision superior along the posterior of the sternum in the substernal location. The distal portion of the lead is introduced into the tunnel via implant tool (e.g., via a sheath). As the distal portion is advanced through the substernal tunnel, the distal portion is relatively straight. The pre-formed or shaped undulating configuration is flexible enough to be straightened out while routing the lead through a sheath or other lumen or channel of the implant tool. Once the distal portion is in place, the implant tool is withdrawn toward the incision and removed from the body of the patient while leaving the lead in place along the substernal path. As the implant tool is withdrawn, the distal end of the lead takes on its pre-formed undulating configuration, and the shield transitions to its deployed configuration.

In some examples, rather than extending in a superior direction along the sternum, the distal portion of the lead may be oriented orthogonal or otherwise transverse to the sternum and/or inferior to the heart. In such examples, the lead may include one or more shields that cover a portion of an outer surface of one or more electrodes, e.g., an anterior and/or inferior portion and/or in a direction relating to an anatomical feature (such as the diaphragm), according to any of the examples described herein. Such shield(s) may impede an electrical field in a direction away from the heart, which may be an anterior inferior, posterior, and/or superior direction. In some examples, the distal portion of the lead may be placed between the heart and lung as well as within the pleural cavity.

In accordance with the techniques of the disclosure, the pacing electrode of electrodes 416 may be configured to decrease the pacing voltage threshold. For example, a conductive surface may be disposed on a shield and electrically coupled to the pacing electrode, which may reduce a resistance of the pacing electrode and/or expand an electric field generated by the pacing electrode. Reducing the resistance of the pacing electrode and/or expanding the electric field generated by the pacing electrode may reduce an amount of current used to generate a pacing pulse, which may decrease an amount of power used by IMD 9.

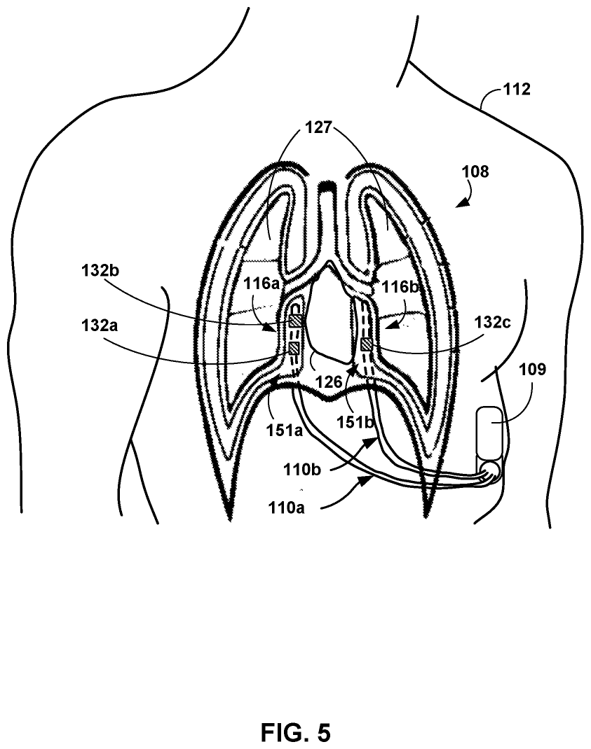

As illustrated in FIGS. 5-9, placement of leads and/or leadless pacing devices within various extracardiac locations may achieve the same or substantially similar effects as compared to the placement described above with respect to FIGS. 1-3. Additionally, locations may include, but are not limited to, the pleural sac or pleural cavity, the pericardial or epicardial regions, or some intrathoracic, non-cardiac/extracardiac vascular locations. Also illustrated are combinations of various pacing devices, including devices with leads and leadless pacing devices, which may be used alone, or in combination with one another.

Device placement variations may include not only location of the device, but also the orientation of the device (e.g., horizontal, vertical, etc.).

FIG. 5 is a conceptual drawing illustrating a front view of a patient 112 implanted with an example extracardiac implantable medical device system 108. System 108 includes an IMD 109 connected to a first lead 110a and a second lead 110b. As shown in the example of FIG. 5, distal portion 116a of lead 110a and distal portion 116b of lead 110b may be placed between the heart 126 and lung 127 within the pleural cavity (e.g., intrapleural placement). Leads 110a and 110b (complete) may be configured as described above with respect to the one or more leads 10 of FIGS. 1-3, except as described herein. Leads 110a and 110b may be configured to be placed in the pleural cavity (e.g., intrapleural placement). In some examples, where lead 110a is configured to position a first subset of the plurality of electrodes (such as electrodes 132a and 132b) to pace the RA and RV, lead 110a may be positioned in right pleural cavity 151a or left pleural cavity 151b (collectively “pleural cavity 151”). Lead 110a may be positioned anterior relative to heart 126. Lead 110a may be positioned lateral relative to heart 126. Lead 110a may be positioned anterolateral relative to heart 126. In some examples, where lead 110b is configured to position a second subset of the plurality of electrodes (such as electrode 132c) to pace the LV, lead 110b may be positioned in right pleural cavity 151a or left pleural cavity 151b. Lead 110b may be positioned posterior relative to heart 126. Lead 110b may be positioned lateral relative to heart 126. Lead 110b may be positioned posterolateral relative to heart 126. In some examples, the first subset of the plurality of electrodes 132 or the second subset of the plurality of electrodes 132 can include one or more electrodes. As shown in the example of FIG. 5, lead 110a may be placed in right pleural cavity 151a anterior to heart 126, and lead 110b may be placed in left pleural cavity 151b posterior to heart 126. However, alternative arrangements of leads 110 may be contemplated. Lead 110a may be placed in left pleural cavity 151b anterior to heart 126, and lead 110b may be placed in right pleural cavity 151a posterior to heart 126. Lead 110a and lead 110b may both be placed in right pleural cavity 151a (one posterior and one anterior to heart 126) or lead 110a and lead 110b may both be placed in left pleural cavity 151b (one posterior and one anterior to heart 126). Lead 110a and lead 110b may share a common point of entry into pleural cavity 151. For example, a single incision site proximate the xiphoid process may facilitate delivery of one or more leads 110 into the pleural cavity. Lead 110a and lead 110b may have different points of entry into pleural cavity 151. For example, two or more incisions proximate the xiphoid process may facilitate delivery of one or more leads 110, such as where lead 110a is placed into the pleural cavity through a first incision, and lead 110b is placed into the pleural cavity through a second incision. For an intercostal approach, a single incision may be made to facilitate delivery of one or more leads 110, or two or more incisions may be made to facilitate delivery of one or more leads 110.

In some examples, placement of one or more leads 110 can occur in a combination of locations within the mediastinum or the pleural cavity. For example, lead 110a may be placed in the mediastinum, while lead 110b is placed in the pleural cavity. As another example, lead 110a may be placed in the pleural cavity, while lead 110b is placed in the mediastinum. In some examples, where lead 110a is placed the mediastinum and lead 110b is placed in the pleural cavity, lead 110a may be placed anterior to heart 126 in the mediastinum and lead 110b may be placed posterior to heart 126 in the pleural cavity. In some examples where lead 110a is placed the pleural cavity and lead 110b is placed in the mediastinum, lead 110a may be placed anterior to heart 126 in the pleural cavity and lead 110b may be placed posterior to heart 126 in the mediastinum.

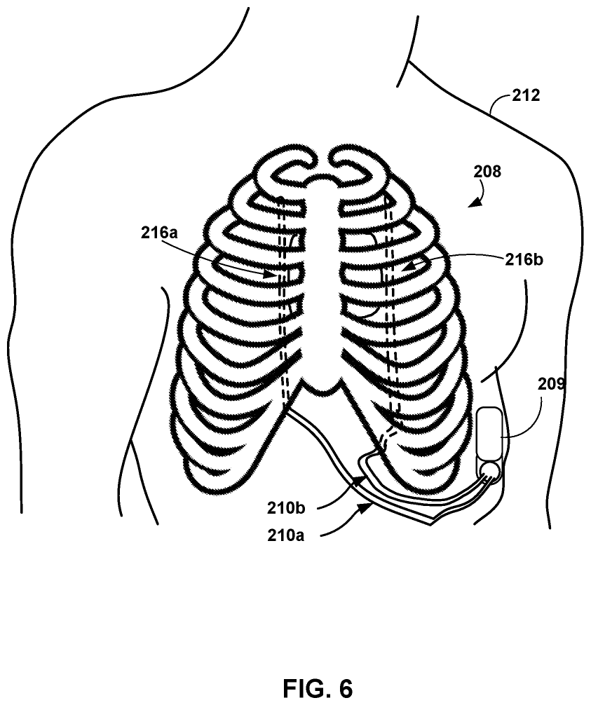

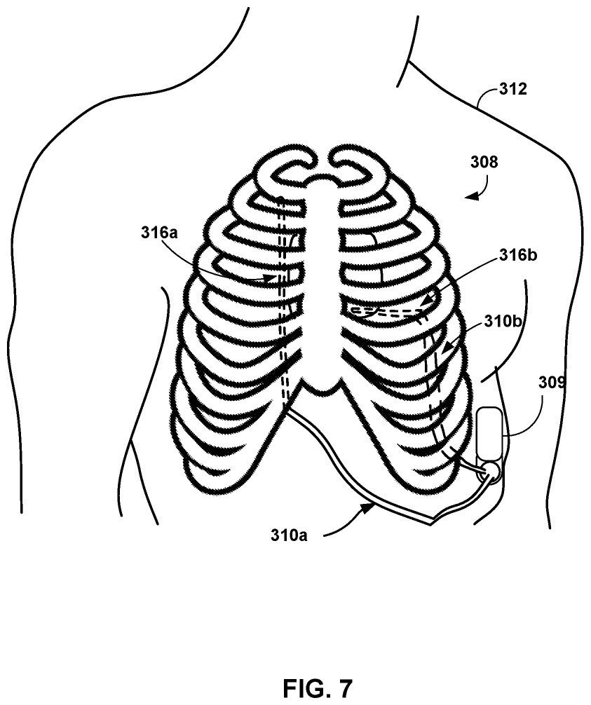

FIG. 6 is a conceptual drawing illustrating a front view of a patient 212 implanted with an example extracardiac implantable medical device system 208 including leads configured to be oriented vertically within patient 212. FIG. 7 is a conceptual drawing illustrating a front view of a patient 312 implanted with an example extracardiac implantable medical device system 308 including leads configured to be oriented vertically and/or horizontally within patient 312. As shown in the examples of FIGS. 6-7, various orientations of the one or more leads may be contemplated, whether one or more leads are placed in the mediastinum or are placed in the pleural cavity. Various orientations of leads may allow for multiple different therapy vectors. System 208 includes an IMD 209 connected to a first lead 210a and a second lead 210b. Leads 210a and 210b (complete) may be configured as described above with respect to the one or more leads 10 of FIGS. 1-3, except as described herein. In the example of FIG. 6, one or more leads 210 may be substantially vertical. Additionally, one or more leads 210 may be substantially parallel. For example, distal portion 216a of lead 210a and distal portion 216b of lead 210b may be vertical with the mediastinum or within the pleural cavity. In this manner, a first lead, such as distal portion 216a of lead 210a and a second lead, such as distal portion 216b of lead 210b, may be substantially parallel. However, one or more leads 210 may also be substantially horizontal.

In the example of FIG. 7, one or more leads 310 may be horizontal or substantially horizontal. System 308 includes an IMD 309 connected to a first lead 310a and a second lead 310b. Leads 310a and 310b (complete) may be configured as described above with respect to the one or more leads 10 of FIGS. 1-3, except as described herein. In some examples, distal portion 316b of lead 310b may be horizontal within the mediastinum or within the pleural cavity. In this example, distal portion 316a of lead 310a may be vertical. In this manner, a first lead, such as distal portion 316a of lead 310a and a second lead, such as distal portion 316b of lead 310b, may be substantially perpendicular. Other orientations and combinations of orientations of one or more leads 310 may be possible. For example, distal portion 316a of lead 310a may be horizontal while distal portion 316b of lead 310b may be vertical. Further, both distal portion 316a of lead 310a and distal portion 316b and lead 310b may be horizontal. In addition to the horizontal and vertical orientations, distal portion 316a of lead 310a and distal portion 316b or lead 310b may be diagonal within a patient 312. Distal portion 316a of lead 310a and distal portion 316b of lead 310b may not be parallel nor perpendicular (e.g., disposed at an angle or askew).

In some examples, one or more leads 310 may be placed horizontally or substantially horizontal in one or more supradiaphragmatic locations. In such examples, one or more leads 310 may be draped over the diaphragm of a patient 312. In such examples, one or more supradiaphragmatic locations may be within the mediastinum of the patient 312. In such examples, one or more pace/sense electrodes on one or more leads 310 may be directed upward toward the heart. In such examples, the electrodes directed upwards toward the heart may be paddle electrodes. In such examples, one or more shields on one or more leads 310 may be directed toward the diaphragm.

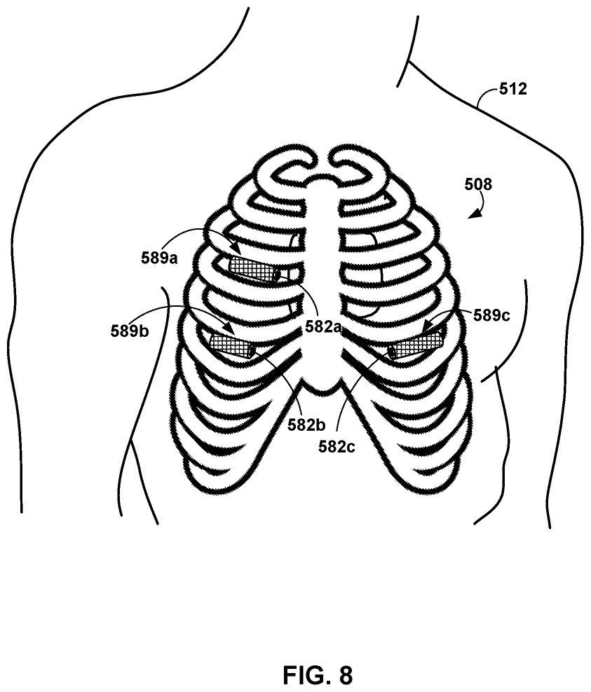

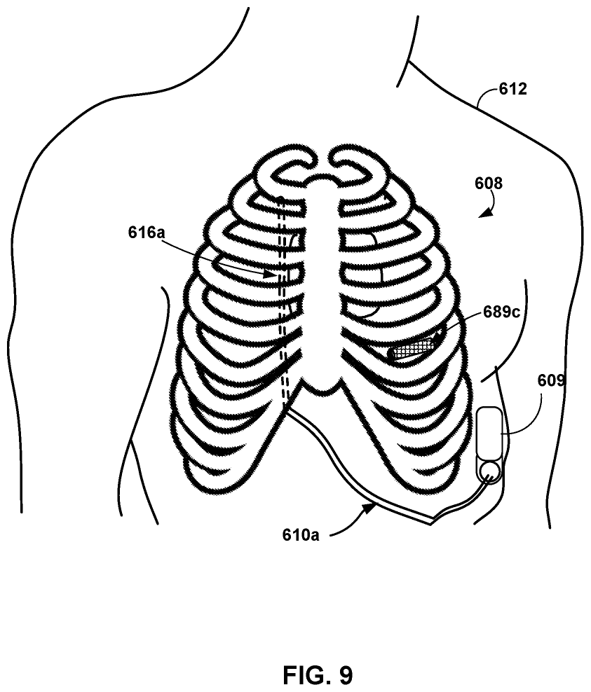

FIG. 8 is a conceptual drawing illustrating a front view of a patient 512 implanted with an example extracardiac implantable medical device system including multiple leadless pacing devices. FIG. 9 is a conceptual drawing illustrating a front view of a patient 612 implanted with an example extracardiac implantable medical device system along with a plurality of leadless pacing devices. As shown in the examples of FIGS. 8-9, leadless pacing systems, including one or more leadless pacing devices may be used in connection with, or instead of, an IMD system. In the example of FIG. 8, leadless pacing system 508 may include a first leadless pacing device 589a, a second leadless pacing device 589b, and a third leadless pacing device 589c (collectively “plurality of leadless pacing devices 589”). The plurality of leadless pacing devices 589 may be configured to be positioned in a plurality of extracardiac locations (such as extravascularly or in one or more extracardiac vessels). In the example of FIG. 8, leadless pacing device 589a includes electrodes 582a, leadless pacing device 589b includes electrodes 582b, and leadless pacing device 589c includes electrodes 582c (collectively referred to as “plurality of electrodes 582”). The plurality of leadless pacing devices may be configured to deliver cardiac pacing to the RA, the RV, the LV, or any combination thereof via plurality of electrodes 582 on the plurality of leadless pacing devices 589. In some examples, the plurality of leadless pacing devices may additionally or alternatively capture the LA along with or instead of the RA, the RV, the LV. In some examples, the cardiac pacing may include cardiac resynchronization therapy (CRT). The plurality of leadless pacing device 589 may be configured to communicate with one another, or with an IMD system. The plurality of leadless pacing devices 489 may be placed in substernal locations substantially similar to the one or more leads previously described. For example, one or more leadless pacing devices of the plurality of leadless pacing devices 589 may be positioned in the mediastinum of the patient 512. One or more leadless pacing devices of the plurality of leadless pacing devices 589 may be positioned in the pleural cavity of the patient 512.

In the example of FIG. 8, all pacing is delivered via the plurality of leadless pacing devices 589. A first subset of leadless pacing devices, which may include leadless pacing device 589a and leadless pacing device 589b, may be positioned to pace the RA and the RV. For example, the first subset of leadless pacing devices, which may include leadless pacing device 589a and leadless pacing device 589b, may be placed in an anterior mediastinum of the patient 512. As another example, the first subset of leadless pacing devices, which may include leadless pacing device 589a and leadless pacing device 589b, may be placed in an anterior pleural cavity of the patient 512. A second subset of leadless pacing devices, which may include leadless pacing device 589c, may be positioned to pace the LV. For example, the second subset of leadless pacing devices, which may include leadless pacing device 589c, may be placed in a posterior mediastinum of the patient 512. As another example, the second subset of leadless pacing devices, which may include leadless pacing device 589c, may be placed in a posterior pleural cavity of the patient 512. The first subset of the plurality of leadless pacing devices 589 may be configured to be positioned in a right pleural cavity and the second subset of the plurality of leadless pacing devices 589 may be configured to be is positioned in a left pleural cavity. The first subset of the plurality of leadless pacing devices 589 may be configured to be positioned in a left pleural cavity and the second subset of the plurality of leadless pacing devices 589 may be configured to be is positioned in a right pleural cavity.

The plurality of leadless pacing devices 589 may be positioned in various orientations, whether the plurality of leadless pacing devices 589 are placed in the mediastinum or are placed in the pleural cavity. One or more leadless pacing devices of the plurality of leadless pacing devices 589 may be substantially vertical. One or more leadless pacing devices of the plurality of leadless pacing devices 589 may be substantially horizontal. Some leadless pacing devices of the plurality of leadless pacing devices 589 may be substantially vertical while other leadless pacing devices of the plurality of leadless pacing devices 589 may be substantially horizontal. In this manner, some leadless pacing devices of the plurality of leadless pacing devices 589 may be substantially parallel while other leadless pacing devices of the plurality of leadless pacing devices 589 may be substantially perpendicular.

As shown in the example of FIG. 8, each leadless pacing device may include electrodes. The plurality of leadless pacing devices 589 may include a plurality of electrodes 582 configured to deliver cardiac pacing. The cardiac pacing delivered by the one or more leadless pacing devices may include cardiac resynchronization therapy (CRT). The plurality of leadless pacing devices 589 may be configured to wirelessly communicate with one another, or with an IMD system. In some examples, a timing sequence may allow coordination between the plurality of leadless pacing devices 589 to delivery therapy. For example, a timing sequency may include following the RA to coordinate the appropriate delay for delivering therapy to the RV and LV. Additionally or alternatively, a timing sequence may include following the LA to coordinate the appropriate delay for delivering therapy to the RV and LV. Additionally or alternatively, a timing sequence may include following the RA for the RV and following the LA for the LV. A coordinated timing sequence may periodically self-correct. In some examples, a timing sequence may include following the RA for every beat. In some examples, a timing sequence may include following the LA for every beat. In some examples, a timing sequence may include following the RA for every beat while only periodically following the LA. In some examples, a timing sequence may include following the LA for every beat while only periodically following the RA.

In the example of FIG. 9, pacing system 608 may include one or more leads 610 (such as lead 610a) and one or more leadless pacing devices 689 (such as leadless pacing device 689c). One or more leadless pacing devices 689 may be used in combination with or instead of one or more leads 610. Lead 610a may be connected to IMD 609. Lead 610a may be configured as described above with respect to the one or more leads 10 of FIGS. 1-3, except as described herein. Leadless pacing device 689c may be configured as described above with respect to the plurality of leadless pacing devices 589 of FIG. 8. Any number of leads having a plurality of electrodes may be used in combination with any number of leadless pacing devices to achieve desired results. In some examples, distal portion 616a of lead 610a may be implanted substantially parallel to leadless pacing device 689c. In some examples, distal portion 616a of lead 610a may be implanted substantially perpendicular to leadless pacing device 689c. In some examples, a leadless pacing device may include an extension segment such that the leadless pacing device can capture both an atrium and ventricle.

As shown in the example of FIG. 9, one or more leads 610 (such as lead 610a) may be configured to position a plurality of electrodes at a plurality of extracardiac locations to deliver cardiac pacing to a first subset of the RA, the RV, and the LV and one or more leadless pacing devices may be configured to deliver cardiac pacing to a second subset of the RA, the RV, and the LV. In some examples, the first subset of the RA, the RV, and the LV may include the RA and the RV, and the second subset of the RA, the RV, and the LV includes the LV. In some examples, the first subset of the RA, the RV, and the LV may include the LV and the second subset of the RA, the RV, and the LV includes the RA and the RV.

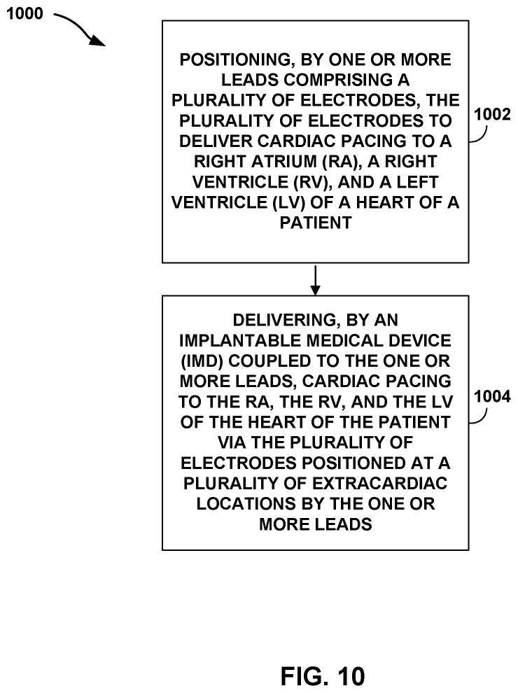

FIG. 10 is a flow chart of an example technique for delivering therapy via an example extracardiac implantable medical device system. The technique of FIG. 10 may be used in connection with any of the devices or systems described in connection with FIGS. 1-9, and is described with respect to system 8 of FIGS. 1-3, as well as system 508 of FIG. 8.

Technique 1000 may include positioning, by one or more leads 10 comprising plurality of electrodes 32, the plurality of electrodes 32 to deliver cardiac pacing to the RA, RV, and LV (1002). In other examples, one or more leads 10 may comprise a plurality of electrodes configured to additionally or alternatively capture the LA along with or instead of the RA, the RV, the LV. As described above, a first lead 10a a may be configured to position a first subset of the plurality of electrodes 32 to deliver cardiac pacing to the RA and the RV, and a second lead 10b may be configured to position a second subset of the plurality of electrodes 32 to deliver cardiac pacing to the LV. The first lead 10a with the first subset of electrodes may be placed in an anterior mediastinum or in an anterior pleural cavity. The second lead 10b with the second subset of electrodes may be placed in a posterior mediastinum or in a posterior pleural cavity. The first lead 10a and second lead 10b may share a common point of entry into the mediastinum or a common point of entry into the pleural cavity. The first lead 10a may be configured to be positioned in a right pleural cavity and the second lead 10b may be configured to be positioned in a left pleural cavity. The first lead 10a may be configured to be positioned in a left pleural cavity and the second lead 10b may be configured to be positioned in a right pleural cavity. The first lead 10a may be positioned perpendicular or parallel to the second lead 10b. In other examples, the technique may include position, by one or more leads 10 comprising plurality of electrodes 32, the plurality of electrodes 32 to deliver cardiac pacing to the LA, LV, and RV.

Technique 1000 may further include delivering, by an IMD coupled to the one or more leads, cardiac pacing to the RA, the RV, and the LV of the heart of the patient via the plurality of electrodes 32 positioned at a plurality of extracardiac positions by the one or more leads 10 (1004). Additionally, the technique may include delivering, by one or more leadless pacing devices 589, cardiac pacing to the RA, the RV, the LV, or any combination thereof. As discussed above, leadless pacing devices 589 can be used in combination with, or instead of, an IMD with one or more leads 10. The leadless pacing devices 589 may be configured to be positioned in the mediastinum or the pleural cavity of the patient. The leadless pacing devices 589 may be configured to be positioned within one or more extracardiac vessels within the patient. The leadless pacing devices 589 may be placed parallel to or perpendicular to other leadless pacing devices or parallel to or perpendicular to the one or more leads 10 of the IMD as discussed above.

In some examples, cardiac pacing delivered to the RA, the RV, and the LV is CRT. In other examples, cardiac pacing that may be delivered to the LA, the LV, and the RV is CRT. In some examples, the plurality of electrodes 32 are configured to deliver cardiac pacing, and one or more electrodes 32 are configured to deliver antitachyarrhythmia shocks. The IMD may include an ICD configured to deliver the antitachyarrhythmia shocks via the one or more electrodes configured to deliver the antitachyarrhythmia shocks.

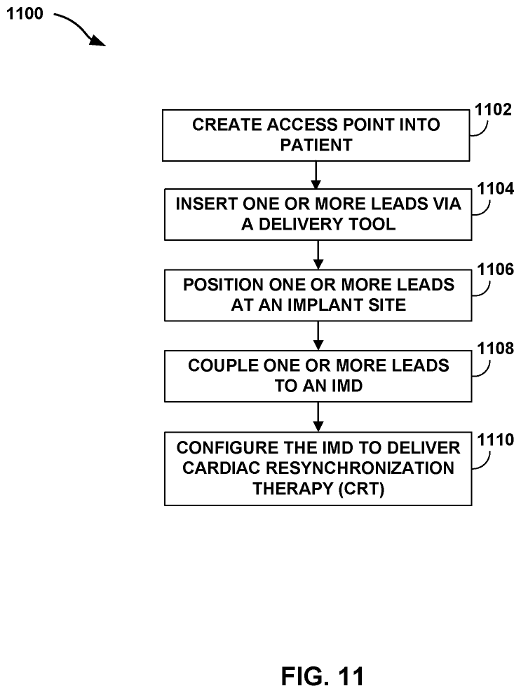

FIG. 11 is a flow chart of an example technique for implanting one or more leads within a patient. The technique of FIG. 11 may be used in connection with any of the devices or systems described in connection with FIGS. 1-9, and is described with respect to system 8 of FIGS. 1-3. The technique may be applicable for implanting one or more leads at one or more extracardiac locations, including, but not limited to, the mediastinum and/or the pleural cavity of a patient. Technique 1100 may include creating an access point into a patient 12 (1102). The access point may include an incision made on the skin/tissue of patient 12 adjacent to or below the xiphoid process. Additionally, the access point may include an incision made on the skin/tissue of patient 12 in an intercostal location, for example, via thoracotomy (i.e., between the ribs). Additionally, the access point may include an incision proximate the manubrium. When accessing via an incision proximate the manubrium, the anterior mediastinum, the posterior mediastinum, the anterior pleural cavity, and/or the posterior pleural cavity may be accessed. The incision may be sized to allow for insertion of a delivery tool and/or tunning tool and navigation of one or more leads 10. In some examples, more than one incision may be made to accommodate one or more leads 10. For example, a first incision may be made to deliver a first lead (such as lead 10a) and a second incision made to deliver a second lead (such as lead 10b).

Continuing with the example of FIG. 11, technique 1100 may include inserting one or more leads 10 into the patient 12 via a delivery tool (1104). The delivery tool may be sized to allow delivery of one or more leads 10 through the delivery tool. An elongated portion of the delivery tool may be advanced within the substernal space of patient 12. The delivery tool may aid in delivering the leads at a location proximate the xiphoid process and/or an intercostal location (i.e., between the ribs). A tunneling instrument may be used in delivery of the one or more leads 10.

Continuing with the example of FIG. 11. technique 1100 may include positioning one or more leads 10 at an implant site (1106). The implant site may include a target location for placement of one or more leads 10. A first lead (such as lead 10a) may have a first target location. In some examples, a first electrode (such as electrode 32a) may be positioned to pace the RV of a patient, such as near the right ventricular center of mass. In some examples, a second electrode (such as electrode 32b) may be positioned to pace the RA of a patient, such as near the right atrial center of mass. In some examples, a third electrode (such as electrode 32c) may be positioned to pace the LV of a patient, such as near the left ventricular center of mass. In some examples, an electrode may be positioned to pace the LA of a patient, such as near the left atrial center of mass. In some examples, electrodes may be placed in other locations relative to the center of mass of the respective atrium or ventricle. In some examples, a first lead (such as lead 10a) may be positioned anterior relative to the heart. A tunneling instrument may direct the first lead (such as lead 10a) to a position anterior relative to the heart. A second lead (such as lead 10b) may have a second target location. In some examples, a second lead (such as lead 10b) may be positioned posterior relative to the heart. A tunneling instrument may direct the second lead (such as lead 10b) to a position posterior relative to the heart. Technique 1100 may include coupling one or more leads to an IMD (1108). In some examples, the IMD may be an ICD. Technique 1100 may include configuring the IMD to deliver therapy, such as cardiac resynchronization therapy (CRT) (1110).

Although the devices, systems, and methods described above relate to cardiac pacing and sensing, the same may be useful for other applications, such as respiration monitoring. For example, placement of devices in intrapleural spaces may be useful for monitoring symptoms of certain respiratory conditions including, but not limited to, diabetes, Chronic Obstructive Pulmonary Disease (COPD), sleep apnea, and heart failure. Sensors on distal portions of leads may be added, modified, or otherwise configured for certain respiratory conditions. Such sensors may include, but are not limited to pressure sensors, glucose sensors, electrodes to measure impedance, an accelerometer to detect physical displacement, and a microphone to detect acoustics. Respiratory monitoring for diabetes may include monitoring glucose levels as a proxy for lung function, and monitoring Kussmaul breathing, which is characterized by deep, rapid and labored breathing, as is present in ketoacidosis. Respiratory monitoring for sleep apnea may include detecting and monitoring effects of therapy, which may include continuous positive airway pressure (CPAP) therapy or hypoglossal nerve stimulation. Respiratory monitoring for heart failure may include monitoring for pulmonary edema and/or Cheyne-Stokes breathing. Respiratory monitoring may also improve the sensitivity and specificity of ventricular tachycardia and ventricular fibrillation (VT/VF) monitoring, since shortness or cessation of breath can corroborate traditional VT/VF monitoring methods. Intrapleural placement of monitoring devices may also be useful for detecting heart sounds via acoustic sensors useful for monitoring heart function and detecting valvular diseases.

Placement of respiratory monitoring devices may use the same or substantially the same techniques as discussed above. Electrodes discussed above (including pace/sense electrodes) may be modified to monitor the respiratory conditions discussed above. For example, the location and orientation of sensors on the leads may be configured to monitor respiratory conditions. For respiratory monitoring, one or more respiratory monitoring devices may be placed in the right, left, or both pleural cavities. Placement of such respiratory monitoring devices may be configured for the given respiratory monitoring application. For example, the orientation of such respiratory monitoring devices may be configured for the given respiratory monitoring application. For example, the devices may be placed vertically, horizontally, or any combination of vertical and horizontal orientations to achieve desired results. Devices may include an implantable medical device coupled to one or more leads configured to monitor one or more respiration parameters, as well as leadless monitoring devices configured to monitor one or more respiration parameters.

In other examples, placement of devices in various substernal or intrapleural spaces may enable atrial fibrillation (AF) monitoring. Depending on position of one or more devices configured to monitor AF, AF monitoring may be performed from the R-wave alone, or from the R-wave with P-wave corroboration. P-wave signals may be observed where the monitoring device is placed superior to the atrial silhouette.

Clause 1. A system comprising: one or more leads comprising a plurality of electrodes, wherein the one or more leads are configured to position the plurality of electrodes at a plurality of extracardiac locations to deliver cardiac pacing to a right atrium (RA), a right ventricle (RV), and a left ventricle (LV) of a heart of a patient; and an implantable medical device (IMD) coupled to the one or more leads, wherein the IMD device is configured to deliver the cardiac pacing via the plurality of electrodes.

Clause 2. The system of clause 1, wherein the one or more leads comprises: a first lead configured to position a first subset of the plurality of electrodes to deliver cardiac pacing to the RA and the RV; and a second lead configured to position a second subset of the plurality of electrodes to deliver cardiac pacing to the LV.

Clause 3. The system of clause 2, wherein the first lead is configured to be positioned in an anterior mediastinum of the patient.

Clause 4. The system of clauses 2 or 3, wherein the second lead is configured to be positioned in a posterior mediastinum of the patient.

Clause 5. The system of any of clauses 2 to 4, wherein the first lead and the second lead are configured to share a common point of entry into a mediastinum of the patient.

Clause 6. The system of clause 2, wherein the first lead is configured to be positioned in a pleural cavity of the patient.

Clause 7. The system of clauses 2 or 6, wherein the second lead is configured to be positioned in a pleural cavity of the patient.

Clause 8. The system of any of clauses 2, or 6 to 7, wherein the first lead is configured to be positioned in a right pleural cavity and the second lead configured to be is positioned in a left pleural cavity.

Clause 9. The system of any of clauses 2, or 6 to 7, wherein the first lead is configured to be positioned in a left pleural cavity and the second lead is configured to be positioned in a right pleural cavity.

Clause 10. The system of any one or more of clauses 6 to 9, wherein the first lead and the second lead are configured to share a common point of entry into the pleural cavity of the patient.

Clause 11. The system of any one or more of clauses 2 to 10, wherein the first lead is configured to be positioned substantially perpendicular to the second lead.

Clause 12. A system comprising: one or more leads comprising a plurality of electrodes, wherein the one or more leads are configured to position a plurality of electrodes at a plurality of extracardiac locations to deliver cardiac pacing to a first subset of a right atrium (RA), a right ventricle (RV), and a left ventricle (LV) of a heart of a patient; an implantable medical device (IMD) coupled to the one or more leads, wherein the IMD device is configured to deliver the cardiac pacing via the plurality of electrodes; and one or more leadless pacing devices, the one or more leadless pacing devices configured to deliver cardiac pacing to a second subset of the RA, the RV, the LV.

Clause 13. The system of clause 12, wherein the one or more leadless pacing devices are configured to be positioned in the mediastinum of the patient.

Clause 14. The system of clause 12, wherein the one or more leadless pacing devices are configured to be positioned in the pleural cavity of the patient.

Clause 15. The system of any one or more of clauses 12 to 14, wherein the one or more leadless pacing devices are configured to be positioned substantially perpendicular to the one or more leads.

Clause 16. The system of any one or more of clauses 1 to 11 or 12 to 15, wherein the cardiac pacing comprises cardiac resynchronization therapy (CRT).

Clause 17. The system of any one or more of clauses 1 to 11 or 12 to 16, wherein the plurality of electrodes comprise a plurality of electrodes configured to deliver cardiac pacing, wherein the one or more leads comprise one or more electrodes configured to deliver antitachyarrhythmia shocks, and wherein the IMD comprises an implantable cardioverter defibrillator (ICD) configured to deliver the antitachyarrhythmia shocks via the one or more electrodes configured to deliver the antitachyarrhythmia shocks.

Clause 18. A method comprising: positioning, by one or more leads comprising a plurality of electrodes, the plurality of electrodes to deliver cardiac pacing to a right atrium (RA), a right ventricle (RV), and a left ventricle (LV) of a heart of a patient; and delivering, by an implantable medical device (IMD) coupled to the one or more leads, cardiac pacing to the RA, the RV, and the LV of the heart of the patient via the plurality of electrodes positioned at a plurality of extracardiac locations by the one or more leads.

Clause 19. The method of clause 18, wherein the one or more leads comprises: a first lead configured to position a first subset of the plurality of electrodes to deliver cardiac pacing to the RA and the RV, and a second lead configured to position a second subset of the plurality of electrodes to deliver cardiac pacing to the LV.

Clause 20. The method of clause 19, wherein the first lead is configured to be positioned in an anterior mediastinum of the patient.

Clause 21. The method of clauses 19 or 20, wherein the second lead is configured to be positioned in a posterior mediastinum of the patient.

Clause 22. The method of any one or more of clauses 19 to 21, wherein the first lead and the second lead are configured to share a common point of entry into a mediastinum of the patient.

Clause 23. The method of clause 19, wherein the first lead is configured to be positioned in a pleural cavity of the patient.

Clause 24. The method of clauses 19 or 23, wherein the second lead is configured to be positioned in a pleural cavity of the patient.

Clause 25. The method of any one or more of clauses 19, or 23 to 24, wherein the first lead is configured to be positioned in a right pleural cavity and the second lead configured to be is positioned in a left pleural cavity.

Clause 26. The method of any one or more of clauses 19, or 23 to 24, wherein the first lead is configured to be positioned in a left pleural cavity and the second lead is configured to be positioned in a right pleural cavity.

Clause 27. The method of any one or more of clauses 23 to 26, wherein the first lead and the second lead are configured to share a common point of entry into the pleural cavity of the patient.

Clause 28. The method of any one or more of clauses 19 to 27, wherein the first lead is configured to be positioned substantially perpendicular to the second lead.

Clause 29. A method comprising: positioning, by one or more leads comprising a plurality of electrodes, the plurality of electrodes to deliver cardiac pacing to a first subset of a right atrium (RA), a right ventricle (RV), and a left ventricle (LV) of a heart of a patient; delivering, by an implantable medical device (IMD) coupled to the one or more leads, cardiac pacing to the first subset of the RA, the RV, and the LV of the heart of the patient via the plurality of electrodes positioned at a plurality of extracardiac locations by the one or more leads; and delivering, by one or more leadless pacing devices, cardiac pacing to a second subset of the RA, the RV, and the LV of a heart of a patient.

Clause 30. The method of clause 29, wherein the one or more leadless pacing devices are configured to be positioned in the mediastinum of the patient.

Clause 31. The method of clause 29, wherein the one or more leadless pacing devices are configured to be positioned in the pleural cavity of the patient.

Clause 32. The method of any one or more of clauses 29 to 31, wherein the one or more leadless pacing devices are configured to be positioned substantially perpendicular to the one or more leads.

Clause 33. The method of any one or more of clauses 18 to 28 or 29 to 32, wherein the cardiac pacing comprises cardiac resynchronization therapy (CRT).

Clause 34. The method of any one or more of clauses 18 to 28 or 29 to 33, wherein the plurality of electrodes comprise a plurality of electrodes configured to deliver cardiac pacing, wherein the one or more leads comprise one or more electrodes configured to deliver antitachyarrhythmia shocks, and wherein the IMD comprises an implantable cardioverter defibrillator (ICD) configured to deliver the antitachyarrhythmia shocks via the one or more electrodes configured to deliver the antitachyarrhythmia shocks.

Clause 35. A system comprising: a plurality leadless pacing devices configured to be positioned in a plurality of extracardiac locations, the plurality of leadless pacing devices configured to pace to a right atrium (RA), a right ventricle (RV), and a left ventricle (LV) of a heart of a patient.

Clause 36. The system of clause 35, wherein the plurality of leadless pacing devices comprises: a first subset of the plurality of leadless pacing devices configured to deliver cardiac pacing to the RA and RV, and a second subset of the plurality of leadless pacing devices configured to deliver cardiac pacing to the LV.

Clause 37. The system of clause 36, wherein the first subset of the plurality of leadless pacing devices is configured to be positioned in an anterior mediastinum of the patient.

Clause 38. The system of clauses 36 or 37, wherein the second subset of the plurality of leadless pacing devices is configured to be positioned in a posterior mediastinum of the patient.

Clause 39. The system of clause 36, wherein the first subset of the plurality of leadless pacing devices is configured to be positioned in a pleural cavity of the patient.

Clause 40. The system of any one or more of clauses 36 or 39, wherein the second subset of the plurality of leadless pacing devices is configured to be positioned in a pleural cavity of the patient.

Clause 41. The system of clause 36, wherein the first subset of the plurality of leadless pacing devices is configured to be positioned in a right pleural cavity and the second subset of the plurality of leadless pacing devices configured to be is positioned in a left pleural cavity.

Clause 42. The system of clause 36, wherein the first subset of the plurality of leadless pacing devices is configured to be positioned in a left pleural cavity and the second subset of the plurality of leadless pacing devices configured to be is positioned in a right pleural cavity.

Clause 43. The system of any one or more of clauses 35 to 42, wherein the cardiac pacing comprises cardiac resynchronization therapy (CRT).

Clause 44. A system comprising: one or more extracardiac leads comprising a plurality of electrodes, wherein the one or more leads comprises: a first extracardiac lead configured to position a first subset of the plurality of electrodes to deliver cardiac pacing to a right atrium (RA) and a right ventricle (RV) of a heart of a patient, and a second extracardiac lead configured to position a second subset of the plurality of electrodes to deliver cardiac pacing to a left ventricle (LV), wherein the plurality of electrodes comprise a plurality of electrodes configured to deliver cardiac pacing, wherein the one or more leads comprise one or more electrodes configured to deliver antitachyarrhythmia shocks; and an implantable medical device (IMD) coupled to the one or more leads, wherein the IMD device is configured to deliver the cardiac pacing via the plurality of electrodes configured to deliver cardiac pacing and/or deliver antitachyarrhythmia shocks via the one or more electrodes configured to deliver the antitachyarrhythmia shocks, wherein the first lead is configured to be positioned anterior relative to the heart of the patient, and the second lead is configured to be positioned posterior relative to the heart of the patient.

It will be appreciated by persons skilled in the art that the present application is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the application, which is limited only by the following claims.

1. A system comprising:
one or more leads comprising a plurality of electrodes, wherein the one or more leads are configured to position the plurality of electrodes at a plurality of extracardiac locations to deliver cardiac pacing to a right atrium (RA), a right ventricle (RV), and a left ventricle (LV) of a heart of a patient; and
an implantable medical device (IMD) coupled to the one or more leads, wherein the IMD is configured to deliver the cardiac pacing via the plurality of electrodes.

2. The system of claim 1, wherein the one or more leads comprises:
a first lead configured to position a first subset of the plurality of electrodes to deliver cardiac pacing to the RA and the RV; and
a second lead configured to position a second subset of the plurality of electrodes to deliver cardiac pacing to the LV.

3. The system of claim 2, wherein the first lead is configured to be positioned in one of an anterior mediastinum of the patient or a pleural cavity of the patient.

4. The system of claim 2, wherein the second lead is configured to be positioned in a posterior mediastinum of the patient.

5. The system of claim 2, wherein the second lead is configured to be positioned in a pleural cavity of the patient.

6. The system of claim 2, wherein the first lead is configured to be positioned in a right pleural cavity and the second lead configured to be is positioned in a left pleural cavity.

7. The system of claim 2, wherein the first lead is configured to be positioned in a left pleural cavity and the second lead is configured to be positioned in a right pleural cavity.

8. The system claim 2, wherein the first lead and the second lead are configured to share a common point of entry into a pleural cavity of the patient.

9. The system of any one or more of claim 2, wherein the first lead is configured to be positioned substantially perpendicular to the second lead.

10. A system comprising:
one or more leads comprising a plurality of electrodes, wherein the one or more leads are configured to position the plurality of electrodes at a plurality of extracardiac locations to deliver cardiac pacing to a first subset of a right atrium (RA), a right ventricle (RV), and a left ventricle (LV) of a heart of a patient;
an implantable medical device (IMD) coupled to the one or more leads, wherein the IMD is configured to deliver the cardiac pacing via the plurality of electrodes; and
one or more leadless pacing devices, the one or more leadless pacing devices configured to deliver cardiac pacing to a second subset of the RA, the RV, the LV.

11. The system of claim 10, wherein the one or more leadless pacing devices are configured to be positioned in a mediastinum of the patient.

12. The system of claim 10, wherein the one or more leadless pacing devices are configured to be positioned in a pleural cavity of the patient.

13. The system of claim 10, wherein the one or more leadless pacing devices are configured to be positioned substantially perpendicular to the one or more leads.

14. The system of claim 1, wherein the cardiac pacing comprises cardiac resynchronization therapy (CRT).

15. The system of claim 1, wherein the plurality of electrodes comprise a plurality of electrodes configured to deliver cardiac pacing, wherein the one or more leads comprise one or more electrodes configured to deliver antitachyarrhythmia shocks, and wherein the IMD comprises an implantable cardioverter defibrillator (ICD) configured to deliver the antitachyarrhythmia shocks via the one or more electrodes configured to deliver the antitachyarrhythmia shocks.

16. A system comprising:
one or more extracardiac leads comprising a plurality of electrodes, wherein the one or more extracardiac leads comprises:
a first extracardiac lead configured to position a first subset of the plurality of electrodes to deliver cardiac pacing to a right atrium (RA) and a right ventricle (RV) of a heart of a patient, and
a second extracardiac lead configured to position a second subset of the plurality of electrodes to deliver cardiac pacing to a left ventricle (LV),
wherein the plurality of electrodes comprise one or more electrodes configured to deliver cardiac pacing, and
wherein the plurality of electrodes comprise one or more electrodes one or more electrodes configured to deliver antitachyarrhythmia shocks; and
an implantable medical device (IMD) coupled to the one or more leads, wherein the IMD is configured to deliver the cardiac pacing via the one or more electrodes configured to deliver cardiac pacing and/or deliver antitachyarrhythmia shocks via the one or more electrodes configured to deliver the antitachyarrhythmia shocks,
wherein the first extracardiac lead is configured to be positioned anterior relative to the heart of the patient, and the second extracardiac lead is configured to be positioned posterior relative to the heart of the patient.

17. The system of claim 16, wherein the first extracardiac lead is configured to be positioned an anterior mediastinum of the patient.

18. The system of claim 16, wherein the second extracardiac lead is configured to be positioned in a posterior mediastinum of the patient.

19. The system of claim 16,
wherein the IMD is configured to deliver the cardiac pacing via the one or more electrodes, and
wherein the cardiac pacing comprises cardiac resynchronization therapy (CRT).

20. The system of claim 16, wherein the first extracardiac lead is configured to be positioned substantially perpendicular to the second extracardiac lead.
